(19) United States
(12) Patent Application Publication
(10) Pub. No.: US 2026/0306565 A1
(43) Pub. Date: Oct. 8, 2026

(54) PROBIOTIC COMPOSITIONS AND THEIR USE IN THE PREVENTION AND/OR TREATMENT OF ORAL CAVITY PATHOLOGIES AND/OR DISORDERS

(71) Applicant: SYNBALANCE S.R.L., ORIGGIO (VA) (IT)

(72) Inventors: Cristiana PIANGIOLINO, ORIGGIO (VA) (IT); Silvia CASTEGNARO, ORIGGIO (VA) (IT); Patrizia MALFA, ORIGGIO (VA) (IT); Diletta Francesca SQUARANTI, ORIGGIO (VA) (IT); Rosella MALANCHIN, ORIGGIO (VA) (IT)

(21) Appl. No.: 19/481,689

(22) PCT Filed: May 9, 2024
(86) PCT No.: PCT/EP2024/062872

§ 371 Date: Nov. 5, 2025

(30) Foreign Application Priority Data
May 9, 2023 (IT) 102023000009276

(51) Int. Cl. A61K 35/747; A61K 35/00; A61P 31/04; A61P 31/10

(52) U.S. Cl. CPC A61K 35/747; A61P 31/04; A61P 31/10; A61K 2035/115

(57) ABSTRACT

This invention relates to probiotic compositions comprising at least one strain selected from Lacticaseibacillus paracasei LPC1082, Lacticaseibacillus paracasei LPC1101, Lacticaseibacillus paracasei LPC1114, optionally in combination with at least one strain selected from Lactiplantibacillus plantarum PBS067 and Lacticaseibacillus rhamnosus LRH020, and at least one excipient and/or physiologically acceptable vehicle, for use in the prevention and/or treatment of pathologies and/or disorders of the oral cavity.

### TECHNICAL FIELD OF THE INVENTION

The present invention concerns probiotic compositions comprising at least one strain selected from Lacticaseibacillus paracasei LPC1082, Lacticaseibacillus paracasei LPC1101 and Lacticaseibacillus paracasei LPC1114, and at least one physiologically acceptable excipient and/or vehicle, for use in the prevention and/or treatment of pathologies and/or disorders of the oral cavity.

### STATE OF THE ART

Oral health is a determining factor for quality of life, essential for well-being, and an integral part of overall health. In the European Union, the socioeconomic burden of oral diseases is considerable: they affect the majority of school-age children and adults, representing 5% of public health expenditure. According to WHO data, approximately 50% of the adult population in Europe has reported oral-related diseases. This primarily includes dental caries in permanent teeth, which affected 33.6% of the population in Europe, nearly 335 million cases in 2019.

The oral microbiota, second only to the gut, is the most diverse, hosting over 700 species of bacteria, as well as fungi, viruses, and protozoa. The mouth, with its various niches, is an exceptionally complex habitat where microorganisms colonize the hard surfaces of teeth and the soft tissues of the oral mucosa. In addition to being the starting point of digestion, the oral microbiota is crucial for preserving both oral and systemic health. The oral bacterial community is dominated by phyla: Firmicutes, Bacteroidetes, Proteobacteria, Actinobacteria, Spirochaetes, and Fusobacteria, representing 96% of identified species. However, defining the precise composition of the oral microbiota is challenging as the mouth is an open system often exposed to external agents through food interaction or social contact.

The oral microbiota typically exists in the form of a biofilm, playing a crucial role in maintaining local homeostasis, protecting the oral cavity, preventing infections, and inhibiting the colonization of pathogens.

The most common oral diseases associated with the oral microbiota are dental caries, periodontal diseases (gingivitis and periodontitis), halitosis, and oral candidiasis. Some of these conditions can escalate into more severe systemic diseases, such as certain carcinogenic forms of the gastrointestinal tract.

### Dental Caries

Dental caries result from an imbalance in the physiological interaction between dental minerals and oral microbial biofilms. Endogenous bacteria such as Streptococcus mutans can produce extracellular carbohydrate polymers like sucrose, allowing them to anchor to the dental surface and contribute to the formation of a solid extracellular matrix that is challenging to eradicate. Additionally, these bacteria have the ability to transport and metabolize a variety of carbon sources, including sugars that contribute to the corrosion of dental plaque.

According to the World Health Organization (WHO), globally, an estimated 2 billion people suffer from permanent tooth decay, while 520 million children would experience decay in their primary teeth. This is primarily attributed to inadequate fluoride exposure (in drinking water and toothpaste), the availability and affordability of high-sugar foods, and limited access to oral healthcare services.

### Periodontal Diseases

Periodontal diseases constitute a group of inflammatory pathologies affecting the periodontium, ultimately leading to tooth loss due to immuno-mediated destruction of periodontal ligaments and dental support structures. The alteration of oral microbiota is associated with diseases actively involved in pathogenesis through the promotion of chronic inflammation. Severe periodontal diseases are estimated to affect approximately 19% of the global adult population, representing over a billion cases worldwide.

Due to bacterial resistance associated with extensive antibiotic prescriptions, innovative alternatives capable of modulating oral microbiota and chronic inflammation through different mechanisms of action than pharmacological therapies are gaining interest.

### Halitosis

Halitosis is a common condition known to be associated with periodontitis, with the putrefactive activity of the lingual microbiota playing a key role in producing volatile malodorous compounds under pathological and physiological conditions.

The first probiotic strain used against halitosis is S. salivarius, which can reduce the concentration of volatile sulphur compounds (VSCs) in breath after pre-treatment with chlorhexidine mouthwash. Additionally, S. salivarius has demonstrated in vitro inhibition of the growth of various oral-associated bacterial species such as Solobacterium moorei, Parvimonas Micra, and Eubacteria sulci.

### Oral Candidiasis

Candida is a significant fungal pathogen causing infections primarily in infants and elderly or immunocompromised individuals. Despite Candida being a member of the oral microbiota, an imbalance in oral equilibrium can favour infections. From this perspective, probiotics can promote a local protective effect against pathogens and indirectly support the host's immune system.

Scientific literature suggests a possible mechanism of action in which certain probiotic lactobacilli, primarily in the urogenital tract, co-aggregate with pathogens, including Candida albicans, preventing adhesion and reducing the microbial load. Additionally, the production of antimicrobial compounds plays a direct role in inhibiting their growth.

Therefore, there is still a need to provide compositions capable of simultaneously preventing and/or treating the most common oral pathologies without the use of antibiotics or pharmacological active ingredients, which could lead to subject drug resistance over time, while maintaining the necessary immunomodulatory, antibacterial, and antimicrobial properties for the prevention and/or treatment of oral cavity pathologies and disorders.

### SUMMARY OF THE INVENTION

The present invention relates to a probiotic composition comprising:
at least one strain selected from Lacticaseibacillus paracasei LPC1082, Lacticaseibacillus paracasei LPC1101 and Lacticaseibacillus paracasei LPC1114, and
at least one physiologically acceptable excipient and/or vehicle, for use in the prevention and/or treatment of pathologies and/or disorders of the oral cavity.

Furthermore, the invention relates to a strain of Lacticaseibacillus paracasei LPC1114, a strain of Lacticaseibacillus paracasei LPC1082, and a strain of Lacticaseibacillus paracasei LPC1101.

Another object of the invention is a probiotic composition comprising Lactiplantibacillus plantarum PBS067 and at least one Lacticaseibacillus paracasei selected from Lacticaseibacillus paracasei LPC1082, Lacticaseibacillus paracasei LPC1101, Lacticaseibacillus paracasei LPC1114; or a probiotic composition comprising Lacticaseibacillus rhamnosus LRH020 and at least one Lacticaseibacillus paracasei selected from Lacticaseibacillus paracasei LPC1082, Lacticaseibacillus paracasei LPC1101, Lacticaseibacillus paracasei LPC1114; or a probiotic composition comprising Lactiplantibacillus plantarum PBS067 and Lacticaseibacillus rhamnosus LRH020 and at least one Lacticaseibacillus paracasei selected from Lacticaseibacillus paracasei LPC1082, Lacticaseibacillus paracasei LPC1101, Lacticaseibacillus paracasei LPC1114; and their use as a medicament.

### BRIEF DESCRIPTION OF FIGURES

FIGS. 1 and 2 display percentage inhibition graphs of the pathogens S. mutans and A. actinomycetemcomitans, respectively. In these figures, LP refers to L. plantarum PBS067, LRH refers to L. rhamnosus LRH020, LPC1114 denotes L. paracasei LPC1114, LPC1082 represents L. paracasei LPC1082, and LPC1101 corresponds to L. paracasei LPC1101.

FIGS. 3A-B depict scanning electron microscopy (SEM) images of dentin infected by A. actinomycetemcomitans after 24 hours of incubation.

FIGS. 4A-B showcase SEM images of dentin after exposure to the probiotic strain of L. paracasei LPC1114 following 24 hours of incubation.

FIGS. 5A-C present SEM images of dentin exposed to the probiotic strain L. paracasei LPC1114 at different incubation times, specifically after 90 minutes, 4 hours, and 15 hours of incubation.

FIGS. 6-7A-E illustrate SEM images of the interaction with A. actinomycetemcomitans with the respective probiotic strains: L. plantarum PBS067, L. rhamnosus LRH020, L. paracasei LPC1114, L. paracasei LPC1082, and L. paracasei LPC1101 after 24 hours of incubation.

### DETAILED DESCRIPTION OF THE INVENTION

The present invention relates to a probiotic composition comprising:
a probiotic combination comprising, or consisting of, at least one strain selected from Lacticaseibacillus paracasei LPC1082, Lacticaseibacillus paracasei LPC1101, Lacticaseibacillus paracasei LPC1114, and
at least one physiologically acceptable excipient,
for use in the prevention and/or treatment of pathologies and/or disorders of the oral cavity.

The probiotic combination may also comprise at least one strain selected from Lactiplantibacillus plantarum PBS067 and Lacticaseibacillus rhamnosus LRH020.

It has surprisingly been found that compositions comprising a probiotic combination of at least one strain selected from Lacticaseibacillus paracasei LPC1082, Lacticaseibacillus paracasei LPC1101, Lacticaseibacillus paracasei LPC1114, optionally in combination with Lactiplantibacillus plantarum PBS067 and/or Lacticaseibacillus rhamnosus LRH020; and probiotic compositions comprising Lactiplantibacillus plantarum PBS067 and/or Lacticaseibacillus rhamnosus LRH020; are effective in preventing and/or treating oral cavity pathologies, specifically those selected from periodontitis, gingivitis, halitosis, dental caries, oral candidiasis, and associated cancerous forms.

The probiotic compositions of the invention represent an alternative approach to management strategies, such as for dental caries. They adhere to oral tissues, preventing the adhesion/colonization/proliferation of cariogenic pathogens, producing inhibitors of cellular adhesion and antibacterial agents. They also contribute to nutrient consumption before cariogenic pathogens can utilize them. Simultaneously, these probiotic compositions address periodontal distress, demonstrating overall benefits, including the reduction of gingival bleeding.

Ultimately, the probiotic compositions of the invention not only aid in maintaining periodontal health but also in preserving a healthy tongue ecology. The posterior dorsal surface of circumvallate papillae, known for hosting a high abundance of odor-associated bacterial species, is targeted, effectively combating halitosis and periodontitis.

According to a preferred embodiment of the invention, the probiotic combination comprises, or consists of, at least two strains selected from Lactiplantibacillus plantarum PBS067, Lacticaseibacillus paracasei LPC1082, Lacticaseibacillus paracasei LPC1101, Lacticaseibacillus paracasei LPC1114, and Lacticaseibacillus rhamnosus LRH020, preferably at least three strains,

wherein at least one strain is selected from Lacticaseibacillus paracasei LPC1082, Lacticaseibacillus paracasei LPC1101 and Lacticaseibacillus paracasei LPC1114.

According to a further embodiment of the invention, the probiotic combination comprises, or consists of, Lactiplantibacillus plantarum PBS067 and at least one strain of Lacticaseibacillus paracasei selected from Lacticaseibacillus paracasei LPC1082, Lacticaseibacillus paracasei LPC1101, Lacticaseibacillus paracasei LPC1114, preferably the probiotic combination comprises, or consists of, Lactiplantibacillus plantarum PBS067 and Lacticaseibacillus paracasei LPC1114.

In another embodiment, the probiotic combination comprises, or consists of, Lactiplantibacillus plantarum PBS067 and/or Lacticaseibacillus rhamnosus LRH020.

In another embodiment, the probiotic combination comprises, or consists of, Lacticaseibacillus rhamnosus LRH020 and at least one strain of Lacticaseibacillus paracasei selected from Lacticaseibacillus paracasei LPC1082, Lacticaseibacillus paracasei LPC1101, Lacticaseibacillus paracasei LPC1114.

In a further embodiment, the probiotic combination comprises, or consists, of Lactiplantibacillus plantarum PBS067, Lacticaseibacillus rhamnosus LRH020, and at least one strain of Lacticaseibacillus paracasei selected from Lacticaseibacillus paracasei LPC1082, Lacticaseibacillus paracasei LPC1101, Lacticaseibacillus paracasei LPC1114.

Preferably, in the probiotic compositions of the invention, at least one strain of Lacticaseibacillus paracasei is Lacticaseibacillus paracasei LPC1114.

The strain of Lactiplantibacillus plantarum (L. plantarum or LP) named “PBS067” has been deposited at DSMZ—Deutsche Sammlung von Mikroorganismen und Zellkulturen GmbH under accession number “DSM 24937” on 17 Jun. 2011 under the Budapest Treaty.

The strain of Lacticaseibacillus rhamnosus (L. rhamnosus or LRH) named “LRH020” has been deposited at DSMZ—Deutsche Sammlung von Mikroorganismen und Zellkulturen GmbH under accession number “DSM 25568” on 17 Jan. 2012 under the Budapest Treaty.

Object of the present invention is the strain of Lacticaseibacillus paracasei (L. paracasei or LPC) named “LPC1114” has been deposited at DSMZ—Deutsche Sammlung von Mikroorganismen und Zellkulturen GmbH under accession number “DSM 34559” on 23 Mar. 2023 under the Budapest Treaty.

Object of the present invention is the strain of Lacticaseibacillus paracasei (L. paracasei or LPC) named “LPC1082” has been deposited at DSMZ—Deutsche Sammlung von Mikroorganismen und Zellkulturen GmbH under accession number “DSM34557” on 23 Mar. 2023 under the Budapest Treaty.

Object of the present invention is the strain of Lacticaseibacillus paracasei (L. paracasei or LPC) named “LPC1101” has been deposited at DSMZ—Deutsche Sammlung von Mikroorganismen und Zellkulturen GmbH under accession number “DSM 34558” on 23 Mar. 2023 under the Budapest Treaty.

The probiotic compositions of the invention further include at least one physiologically acceptable excipient and/or vehicle.

These excipients and/or physiologically acceptable vehicles are preferably selected from the group comprising, or consisting of, maltodextrin, magnesium stearate, corn starch, hydroxypropyl methylcellulose (HPMC), vitamin D3, vitamin C, vitamin A, mannitol, fructo-oligosaccharides (FOS), GOS, xylitol, calcium, fluoride, magnesium, and collagen.

The compositions can be formulated by conventional methods. Preferred administration forms are solid formulations, such as hard capsules, sachets, oro-soluble sticks, tablets, chewing gums, candies, or liquid formulations, such as vials with a single- or multi-dose dispensing cap, multi-dose dispersions in oily phase, drops, syrups, multi-phase emulsions, sprays, gels, etc.

When the administration form is a multi-phase emulsion, in the solid phase, the physiologically acceptable excipients and/or vehicles are preferably selected from the group consisting of or consisting in vitamin D3, magnesium stearate, and maltodextrin, while in the liquid phase, the physiologically acceptable excipients and/or vehicles are preferably selected from the group consisting of or consisting in water, potassium sorbate, sodium benzoate, inulin, fructo-oligosaccharides (FOS), flavour, citric acid, sucralose, calcium citrate, and vitamin C.

Lactiplantibacillus plantarum (LP), or the strain of Lactiplantibacillus plantarum PBS067, may be present in a weight percentage on the total weight of the probiotic composition from 0.5 to 25% w/w, preferably from 0.7 to 10% w/w, more preferably from 1 to 5% w/w.

Lactiplantibacillus plantarum (LP), or the strain of Lactiplantibacillus plantarum PBS067, may be present in each single unit dose in a variable quantity from 0.5 to 2.5 billion CFU, preferably in a quantity variable from 1 to 2 billion CFU, more preferably in a quantity equal to 1 billion CFU.

Lacticaseibacillus rhamnosus (LRH), or the strain of Lacticaseibacillus rhamnosus LRH020, may be present in the composition in a weight percentage on the total weight of the combination from 0.5 to 25% w/w, preferably from 1 to 10% w/w, more preferably from 1 to 8% w/w.

Lacticaseibacillus rhamnosus (LRH), or the strain of Lacticaseibacillus rhamnosus LRH020, may be present in each single unit dose in a variable quantity from 0.5 to 2.5 billion CFU, preferably in a quantity variable from 1 to 2 billion CFU, more preferably in a quantity equal to 1 billion CFU.

Lactiplantibacillus paracasei (LPC), or the strains of Lactiplantibacillus paracasei LPC1082, LPC1101, and LPC1114, preferably the strain of Lactiplantibacillus paracasei LPC1114, may be present in the composition in a weight percentage on the total weight of the probiotic composition from 0.5 to 25% w/w, preferably from 0.7 to 10% w/w, more preferably from 1 to 5% w/w.

Lactiplantibacillus paracasei (LPC), preferably the strain of Lactiplantibacillus paracasei LPC1114, may be present in each single unit dose in a quantity ranging from 0.5 to 2.5 billion CFU, preferably in a quantity ranging from 1 to 2 billion CFU, and more preferably is present in a quantity equal to 1 billion CFU.

In a preferred embodiment, the combination of the invention comprises, or consists of, the following strains:
Lactiplantibacillus plantarum PBS067, and
at least one strain of L. paracasei selected from L. paracasei LPC1082, L. paracasei LPC1101, L. paracasei LPC1114.

In another preferred embodiment, the combination of the invention comprises, or consists of, the following strains:
Lacticaseibacillus rhamnosus LRH020, and at least one strain of L. paracasei selected from L. paracasei LPC1082, L. paracasei LPC1101, L. paracasei LPC1114.

In another preferred embodiment, the combination of the invention comprises, or consists of, the following strains:
Lactiplantibacillus plantarum PBS067, Lacticaseibacillus rhamnosus LRH020, and
at least one strain of L. paracasei selected from L. paracasei LPC1082, L. paracasei LPC1101, L. paracasei LPC1114.

In a further preferred embodiment, the combination of the invention comprises, or consists of, the following strains:
Lactiplantibacillus plantarum PBS067, and
Lacticaseibacillus rhamnosus LRH020.

The invention also relates to the preferred combinations for use as a medicament.

The strains belonging to the species Lactiplantibacillus plantarum and Lacticaseibacillus paracasei may be present in a 1:1 ratio expressed in CFU, and in a 1:1:1 ratio when a strain of the species Lacticaseibacillus rhamnosus is also present.

The invention relates to probiotic compositions for use in the prevention and/or treatment of oral cavity pathologies and/or disorders.

In a preferred embodiment, the oral cavity pathologies and/or disorders are selected from the group comprising, or consisting of, periodontitis, gingivitis, halitosis, dental caries, oral candidiasis, and cancerous forms derived from them.

Another embodiment of the present invention is a method comprising the administration of a composition according to the invention for the prevention and/or treatment of oral cavity pathologies and/or disorders in a subject in need thereof.

The probiotic compositions of the invention proved to be effective in the prevention and/or treatment of oral cavity pathologies and/or disorders caused by A. actinomycetemcomitans and S. mutans.

The probiotic compositions of the invention comprising a probiotic combination comprising, or consisting of, at least one strain selected from Lacticaseibacillus paracasei LPC1082, Lacticaseibacillus paracasei LPC1101, Lacticaseibacillus paracasei LPC1114, shown to be effective in the prevention and/or treatment of oral cavity pathologies and/or disorders also caused by Candida albicans.

The probiotic compositions of the invention may be administered orally, buccal, sublingually, or topically. Preferably, said compositions are administered orally.

The compositions of the invention may be advantageously used as a medicament in pharmaceutical products, medical devices, dietary supplements, and foods in the form of hard capsules, sachets, oro-soluble sticks, tablets, chewing gums, candies, vials with a single- or multi-dose dispensing cap, multi-dose dispersions in oily phase, drops, syrups, multi-phase emulsions, sprays, gels, etc.

The examples below further illustrate the invention.

### EXAMPLES

### Formulative Example 1

A powder composition in stick has been prepared, and the quantities of each ingredient are shown in Table 1.

TABLE 1

| | % | Billion |
|---|---|---|
| Ingredients | p/p | (CFU/dose) |
| L. plantarum PBS067 | 2.00 | 1 |
| L. paracasei LPC1114 | 2.00 | 1 |
| Mannitol | 10.00 | — |
| FOS | 80.00 | — |
| Maltodextrin | 8.00 | — |

### Formulative Example 2

A powder composition in capsule has been prepared, and the quantities of each ingredient are shown in Table 2.

TABLE 2

| | % | Billion |
|---|---|---|
| Ingredients | p/p | (CFU/dose) |
| L. plantarum PBS067 | 1.00 | 1 |
| L. paracasei LPC1114 | 1.00 | 1 |
| L. rhamnosus LRH020 | 1.67 | 1 |
| Vitamin D3 | 0.12 | — |
| Mannitol | 10.00 | — |
| FOS | 80.01 | — |
| Maltodextrin | 6.20 | — |

### Formulative Example 3

A multi-phase emulsion in single-dose vial with dispensing cap has been prepared, and the quantities of each ingredient are shown in Table 3.

TABLE 3

| | | |
|---|---|---|
| | % | Billion |
| Ingredients | p/p | (CFU/dose) |

Solid phase

| | | |
|---|---|---|
| L. plantarum PBS067 | 4.01 | 1 |
| L. paracasei LPC1114 | 4.01 | 1 |
| Vitamin D3 | 0.02 | — |
| Magnesium stearate | 1.00 | — |
| Maltodexstrin | 90.96 | — |

Liquid phase

| | | |
|---|---|---|
| Water | 98.58 | — |
| Potassium sorbate | 0.1 | — |
| Sodium benzoate | 0.1 | — |
| Inulin | 0.5 | — |
| FOS | 0.5 | — |
| Flavour | 0.1 | — |
| Citric acid | 0.1 | — |
| Sucralose | 0.03 | — |

### Formulative Example 4

A multi-phase emulsion in single-dose vial with dispensing cap has been prepared, and the quantities of each ingredient are shown in Table 4.

TABLE 4

| | | |
|---|---|---|
| | % | Billion |
| Ingredients | p/p | (CFU/dose) |

Soldi phase

| | | |
|---|---|---|
| L. plantarum PBS067 | 4.01 | 1.000 |
| L. paracasei LPC1114 | 4.01 | 1.000 |
| L. rhamnosus LRH020 | 6.69 | 1.000 |
| Magnesium stearate | 1.00 | — |
| Maltodextrin | 84.29 | — |
| Total | 100 | |

Liquid Phase

| | | |
|---|---|---|
| Water | 92.43 | — |
| FOS | 1.00 | — |
| Calcium citrate | 5.69 | — |
| Potassium sorbate | 0.10 | — |
| Sodium benzoate | 0.10 | — |
| Flavour | 0.10 | — |
| Citric acid | 0.10 | — |
| Vitamin C | 0.46 | — |
| Sucralose | 0.02 | — |

### Formulative Example 5

A powder composition in stick has been prepared, and the quantities of each ingredient are shown in Table 5.

TABLE 5

| | % | Billion |
|---|---|---|
| Ingredients | p/p | (CFU/dose) |
| L. paracasei LPC1114 | 0.99 | 1 |
| Vitamin A | 0.16 | — |
| Sorbitol | 79.08 | — |
| FOS | 19.77 | — |

### Formulation Example 6

A powder composition in stick has been prepared, and the quantities of each ingredient are shown in Table 6.

TABLE 6

| | % | Billion |
|---|---|---|
| Ingredients | p/p | (CFU/dose) |
| L. plantarum PBS067 | 0.99 | 1 |
| Vitamin D3 | 0.12 | — |
| Sorbitol | 59.34 | — |
| FOS | 39.56 | — |

### Experimental Examples

The following experimental examples pertain to the evaluation of the efficacy of the compositions of the invention in preventing and/or treating oral cavity disorders. In vitro experiments were conducted to assess the efficacy of probiotic strains, specifically L. plantarum PBS067, L. rhamnosus LRH020, L. paracasei LPC1082, L. paracasei LPC1101, and L. paracasei LPC1114, in preventing and/or treating oral cavity disorders.

### Example 7—Antimicrobial Activity Against A. actinomycetemcomitans and S. mutans

It has been observed that infections by A. actinomycetemcomitans and S. mutans are common in oral cavity-associated pathologies, and specifically, A. actinomycetemcomitans is one of the bacteria responsible for periodontitis, and S. mutans is one of the bacteria responsible for oral caries.

Therefore, to determine the strain-specific activity, each probiotic strain of the invention underwent:
a test using a “preventive model,” where the probiotic is added before the addition of the tested pathogen, namely A. actinomycetemcomitans (AA) or S. mutans (SM), and
a test using a “treatment model,” where the probiotic strain is added simultaneously with the pathogen to be tested, namely A. actinomycetemcomitans (AA) and S. mutans (SM).

To support the actual efficacy of the probiotics in the compositions of the invention, the data were compared with a control, namely acidified culture medium (MRS pH 4.5).

In addition, the combination of L. paracasei LPC1114+L. plantarum PBS067 was tested against S. mutans (SM).

The results are shown in Table 7.

TABLE 7

| | A. actinomycetemcomitans (AA) | | S. mutans (SM) | |
|---|---|---|---|---|
| | Treatment | Preventive | Treatment | Preventive |
| Inhibition | model | model | model | model |
| L. plantarum PBS067 | +++ | ++++ | ++ | +++ |
| L. rhamnosus LRH020 | ++ | +++ | ++ | ++++ |
| L. paracasei LPC1082 | ++ | +++ | ++ | +++ |
| L. paracasei LPC1101 | ++ | +++ | ++ | ++++ |
| L. paracasei LPC1114 | ++ | ++++ | ++ | ++++ |
| L. paracasei LPC1114 + | n.a. | n.a. | ++ | +++++ |
| L. plantarum PBS067 | | | | |
| Control (MRS pH 4.5) | − | − | − | − |

In Table 7, the following indications are provided: ++++ very strong inhibition (halo diameter in a range above 45 mm), +++ strong inhibition (halo diameter in a range from 31 to 45 mm), ++ moderate inhibition (halo diameter in a range from 16 to 30 mm), + slight inhibition (halo diameter in a range from 1 to 15 mm), and − no inhibition.

From Table 7, it is evident that all tested strains exhibited in-vitro antimicrobial activity against the pathogens A. actinomycetemcomitans and S. mutans. In contrast, the control showed no activity, confirming that the observed effect is not related to the presence of acidic pH. Specifically, strains L. plantarum PBS067, L. rhamnosus LRH020, and L. paracasei LPC1114 demonstrated the best in-vitro antimicrobial activity against A. actinomycetemcomitans and S. mutans.

Furthermore, the results in Table 7 highlighted that all tested strains, especially those of L. plantarum PBS067, L. rhamnosus LRH020, and L. paracasei LPC1114, exhibited significant efficacy in both the “preventive model” and the “treatment model,” confirming their effectiveness in both preventing and treating oral cavity disorders such as periodontitis and oral caries.

Moreover, from Table 7 it is possible to note also the behaviour of the combination of L. paracasei LPC1114 and L. plantarum PBS067 which demonstrate a very strong inhibition against S. mutans in the preventive model.

### Example 8—Antimicrobial Activity Against A. actinomycetemcomitans and S. mutans of the Invention Versus Prior Art

The same experimental protocol as in Example 7 was carried out for the strain of the invention, specifically L. rhamnosus LRH020, compared to the well-known strain of the same species, namely L. rhamnosus LGG, and the results are shown in Table 8.

TABLE 8

| | A. actinomycetemcomitans (AA) | | S. mutans (SM) | |
|---|---|---|---|---|
| | Treatment | Preventive | Treatment | Preventive |
| Inhibition | model | model | model | model |
| L. rhamnosus | ++ | +++ | ++ | ++++ |
| LRH020 | | | | |
| L. rhamnosus | + | + | + | ++ |
| LGG | | | | |

From Table 8, it can be observed that the strain of the invention exhibits better antimicrobial activity against the pathogens A. actinomycetemcomitans and S. mutans compared to the well-known art strain LGG, confirming its increased efficacy in both preventing and treating oral disorders such as periodontitis and dental caries.

### Example 9—Evaluation of the Inhibition Rate of A. actinomycetemcomitans and S. mutans

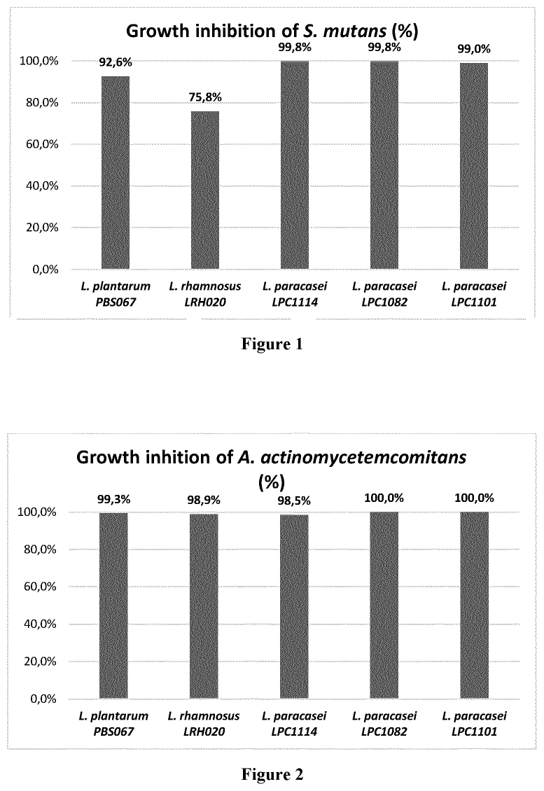

The percentage inhibition of the pathogens A. actinomycetemcomitans and S. mutans was also evaluated in an in-vitro model of pathogen/probiotic co-culture, assessing their survival expressed in CFU for each strain of the compositions of the invention, as highlighted in FIGS. 1 and 2. From FIGS. 1 and 2, it emerges that all the tested strains, particularly L. plantarum PBS067, L. rhamnosus LRH020, L. paracasei LPC1114, proved to be highly effective in inhibiting the growth of both pathogens responsible for oral disorders such as periodontitis and dental caries.

### Example 10—Evaluation of the Antimicrobial Activity of L. paracasei LPC1114 on Dentin Substrate Infected by A. Actinomycetemcomitans

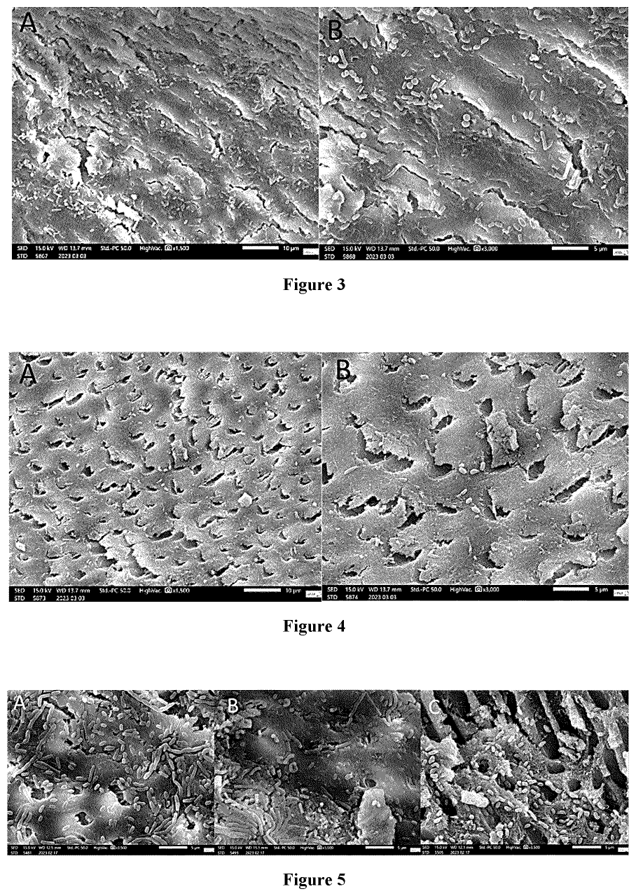

The antimicrobial activity of the L. paracasei LPC1114 probiotic on dentin infected by A. actinomycetemcomitans was evaluated. As evident from FIGS. 3A-B and FIGS. 4A-B, the presence of metabolites produced by the probiotic significantly reduces the concentration of the pathogen present, as shown in the images captured by SEM (scanning electron microscopy). In particular, FIGS. 3A-B depict dentin infected by A. actinomycetemcomitans after 24 hours of incubation, while FIGS. 4A-B show the effect of the L. paracasei LPC1114 probiotic on the A. actinomycetemcomitans pathogen after 24 hours of incubation, i.e., the lower presence of pathogenic cells compared to FIGS. 3A-B.

### Example 11—Evaluation of the Probiotic L. paracasei LPC1114 on the Vitality of A. actinomycetemcomitans

The viability of the A. actinomycetemcomitans pathogen alone (T24, control) or in the presence of metabolites from the L. paracasei LPC1114 probiotic after 24 hours of incubation (T24) was assessed using the Alamar Blue assay, capable of evaluating the metabolic activity of the pathogen. The results are reported in Table 9 through the percentage values of vitality, measured with a colorimetric test at 590 nm.

TABLE 9

| | Vitality |
|---|---|
| Time (hours) | A. actinomycetemcomitans (%) |
| T₂₄ (control, without | 100 |
| L. paracasei LPC1114) | |
| T₂₄ (after adding | 20.94 |
| L. paracasei LPC1114) | |

From Table 9, it can be observed that, following the addition of metabolites produced by the probiotic of the invention compositions, namely L. paracasei LPC1114, the cellular vitality of the A. actinomycetemcomitans pathogen is reduced by 80%, thus demonstrating the high efficacy of said probiotic in countering the proliferation of the pathogen.

### Example 12—Evaluation of Adhesion Activity of L. paracasei LPC1114 on Dentin

The adhesive capacity of the L. paracasei LPC1114 probiotic on dentin was evaluated at various incubation times, specifically after 90 minutes, 4 hours, and after 15 hours of incubation. From the electron microscope images in FIGS. 5A-C, it is possible to observe how the probiotic strain is capable of adhering to the surface and, by wedging into the present irregularities, progressively forming a protective biofilm on the dentin.

### Example 13—Anti-Adhesive Activity of the Probiotic Strains from the Compositions of the Invention Against A. Actinomycetemcomitans and S. mutans Pathogens

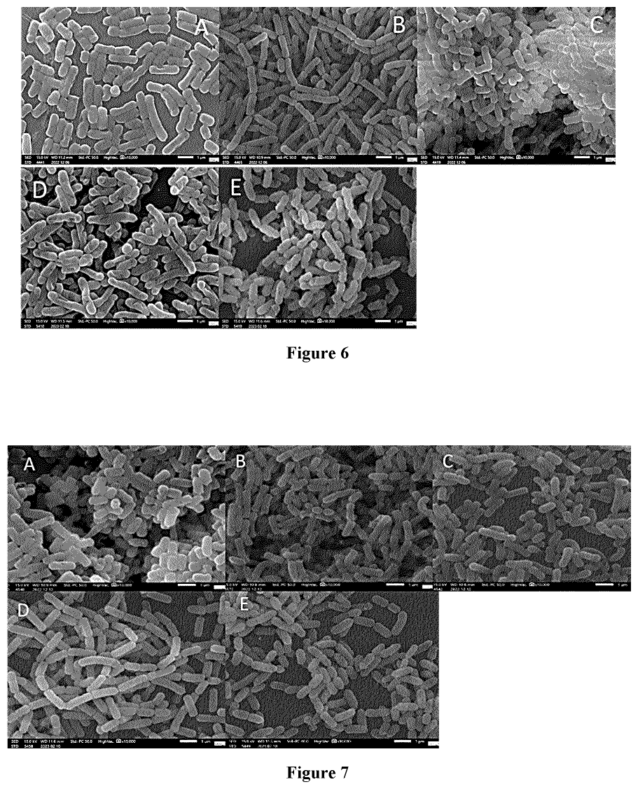

The anti-adhesive activity of the probiotic strains L. plantarum PBS067, L. rhamnosus LRH020, L. paracasei LPC1114, L. paracasei LPC1101, and L. paracasei LPC1082 against the oral pathogens A. actinomycetemcomitans and S. mutans was also evaluated. From the images in FIGS. 6-7A-E, it is evident that the utilized probiotic strains can cover the surface, forming a biofilm capable of preventing the adhesion of pathogens.

### Example 14—Evaluation of the Antimicrobial Activity of L. paracasei LPC1114 Against Candida albicans

It has been observed that infections by Candida albicans are common in oral cavity-associated pathologies.

Therefore, the probiotic strain of the invention, L. paracasei LPC1114, underwent:
a test using a “preventive model,” where the probiotic is added before the addition of the tested pathogen, namely Candida albicans, and
a test using a “treatment model,” where the probiotic strain is added simultaneously with the pathogen to be tested, namely Candida albicans.

The same experimental protocol as in Example 7 was carried out for the strain of the invention, specifically L. paracasei LPC1114, in order to evaluate its efficacy against C. albicans.

The results are shown in Table 10.

TABLE 10

| Stain | 0 h | 48 h |
|---|---|---|
| L. paracasei | + | +++ |
| LPC1114 | (15) | (38) |
| L. plantarum | + | ++ |
| PBS067 | (9) | (23) |
| (reference) | | |

Legend: + inhibition zone diameter 1-15 mm, ++ inhibition zone diameter 16-30 mm, +++ inhibition zone diameter 31-45 mm, ++++ inhibition zone diameter >45 mm

From Table 10, it is evident that the tested strain of L. paracasei LPC1114 exhibited in-vitro antimicrobial activity against the pathogen Candida albicans, higher than that shown by the reference strain of L. plantarum PBS067.

Furthermore, the results in Table 10 highlighted that the tested strain of L. paracasei LPC1114 exhibited significant efficacy in both the “preventive model” and the “treatment model,” confirming their effectiveness in both preventing and treating oral cavity disorders.

1. A method for prevention and/or treatment of a disease and/or disorder of the oral cavity in a subject in need thereof comprising administering to the subject an effective amount of a probiotic composition comprising:
a probiotic combination comprising at least one strain selected from Lacticaseibacillus paracasei LPC1082, Lacticaseibacillus paracasei LPC1101, and Lacticaseibacillus paracasei LPC1114, and
at least one physiologically acceptable excipient and/or vehicle.

2. The method according to claim 1, the probiotic composition further comprising Lactiplantibacillus plantarum PBS067.

3. The method according to claim 1, the probiotic composition further comprising Lacticaseibacillus rhamnosus LRH020.

4. The method according to claim 1, the probiotic composition comprising Lactiplantibacillus plantarum PBS067 and the Lacticaseibacillus paracasei LPC1114.

5. The method according to claim 1, the probiotic composition further comprising Lactiplantibacillus plantarum PBS067, Lacticaseibacillus rhamnosus LRH020.

6. The method according to claim 5, comprising the Lacticaseibacillus paracasei LPC1114.

7. The method according to claim 1, wherein the disease and/or disorder of the oral cavity is selected from the group comprising periodontitis, gingivitis, halitosis, dental caries, oral Candida and cancerous forms thereof.

8. The method according to claim 1, wherein the disease and/or disorder of the oral cavity is caused by A. actinomycetemcomitans, S. mutans and/or Candida albicans.

9-12. (canceled)

13. A probiotic composition comprising:
a probiotic combination comprising at least one strain selected from Lacticaseibacillus paracasei LPC1082, Lacticaseibacillus paracasei LPC1101, Lacticaseibacillus paracasei LPC1114, and
at least one physiologically acceptable excipient and/or vehicle,
for use as a medicament.

14. The probiotic composition of claim 13 comprising the Lacticaseibacillus paracasei LPC1114 and wherein the Lacticaseibacillus paracasei LPC1114 is strain LPC1114 deposited at DSMZ—Deutsche Sammlung von Mikroorganismen und Zellkulturen GmbH under accession number “DSM 34559” on 23 Mar. 2023.

15. The probiotic composition of claim 13 comprising the Lacticaseibacillus paracasei LPC1082 and wherein the Lacticaseibacillus paracasei LPC1082 is strain LPC1082 deposited at DSMZ—Deutsche Sammlung von Mikroorganismen und Zellkulturen GmbH under accession number “DSM34557” on 23 Mar. 2023.

16. The probiotic composition of claim 13 comprising the Lacticaseibacillus paracasei LPC1101 and wherein the Lacticaseibacillus paracasei LPC1101 is strain LPC1101 deposited at DSMZ—Deutsche Sammlung von Mikroorganismen und Zellkulturen GmbH under accession number “DSM34558” on 23 Mar. 2023.

17. The probiotic composition of claim 13 comprising the Lacticaseibacillus paracasei LPC1114 and further comprising Lactiplantibacillus plantarum PBS067.
